(19) United States
(12) Patent Application Publication
(10) Pub. No.: US 2025/0002542 A1
(43) Pub. Date: Jan. 2, 2025

(54) Insect Toxin Delivery Mediated by a Begomovirus Coat Protein

(71) Applicant: UNIVERSITY OF FLORIDA RESEARCH FOUNDATION, INC., Gainesville, FL (US)

(72) Inventors: Bryony C. Bonning, Gainesville, FL (US); Jane E. Polston, Gainesville, FL (US); Jaime Jimenez, Gainesville, FL (US)

(73) Assignee: UNIVERSITY OF FLORIDA RESEARCH FOUNDATION, INC., Gainesville, FL (US)

(21) Appl. No.: 18/701,456

(22) PCT Filed: Oct. 17, 2022
(86) PCT No.: PCT/US22/78221

§ 371 Date: Apr. 15, 2024

Related U.S. Application Data
(60) Provisional application No. 63/257,280, filed on Oct. 19, 2021.

(51) Int. Cl. C07K 14/005; A01N 63/50; A01P 7/04; C07K 5/11; C07K 14/435; C12N 15/82

(52) U.S. Cl. CPC C07K 14/005; A01N 63/50; A01P 7/04; C07K 5/1019; C07K 14/43518; C07K 14/43563; C12N 15/8286; C12N 2750/12022

(57) ABSTRACT

Described herein are fusion proteins comprising a carrier protein attached to a second peptide via a peptide linker, wherein the carrier protein is derived from a tomato yellow leaf curl virus (TYLCV).

### STATEMENT OF GOVERNMENT SUPPORT

This invention was made with government support under 2019-67030-29669 awarded by United States Department of Agriculture, NIFA. The government has certain rights in the invention.

### FIELD OF THE INVENTION

The disclosure relates generally to the field of insect pest control.

### INCORPORATION BY REFERENCE OF SEQUENCE LISTING

This application contains a Sequence Listing in computer readable form (filename: 57278_Seqlisting.XML; 34,390 bytes; created Oct. 15, 2022), which is incorporated herein by reference in its entirety and forms part of the disclosure.

### BACKGROUND

Whitefly-mediated damage of crops, which is often associated with swarms feeding on a wide variety of plants and with virus transmission, results in billions of dollars in crop losses worldwide. Management of the whitefly, Bemisia tabaci has been among the most significant challenges faced by agriculture in the U.S. and elsewhere, following the global expansion of begomoviral disease (Fauquet et al., 2008). Members of the B. tabaci species complex are the sole vectors of begomoviruses, which are some of the most destructive plant pathogens in the world. Management of B. tabaci populations and associated viral plant disease is exacerbated by the widespread resistance of B. tabaci to most of the insecticides in use against this pest (Castle et al., 2010; Palumbo et al., 2001). Despite the tools currently available for B. tabaci management, which include selective insecticides, biological control agents, host plant resistance and physical/mechanical methods, additional commercially viable control measures are needed.

### SUMMARY

In one aspect, described herein is a fusion protein comprising a carrier protein attached to a second peptide via a peptide linker, wherein the carrier protein is derived from a Tomato yellow leaf curl virus (TYLCV).

Compositions comprising the fusion protein described herein and a suitable carrier or excipient are also contemplated.

In another aspect, described herein is a a transgenic plant comprising a nucleotide sequence encoding a fusion protein described herein.

In another aspect, described herein is a transgenic plant comprising the nucleotide sequence set forth in SEQ ID NO: 11.

In another aspect, described herein is a method of producing a transgenic plant comprising introducing a nucleotide encoding the fusion protein described herein or a vector comprising a nucleotide described herein into a cell of the plant.

In another aspect, described herein is a method of producing a transgenic plant comprising introducing the nucleotide sequence of SEQ ID NO: 11 into a cell of the plant.

In another aspect, described herein is a method of controlling insect pests, the method comprising feeding an insect with a food source comprising a fusion protein described herein or a composition described herein, wherein upon ingestion, the fusion protein passes across the gut epithelium and moves into the hemocoel of the insect.

### DETAILED DESCRIPTION

Insect pests continue to reduce crop yields. With resistance to classical chemical insecticides developing in many cases, novel alternative approaches to insect pest control are needed. Although gut-active toxins such as those derived from Bacillus thuringiensis (Bt) have been successfully used for insect pest management, a diverse range of insect-specific insecticidal peptides remains an untapped resource for pest management efforts. These toxins act within the insect hemocoel (body cavity) rather than in the gut and hence require a delivery system to access their target site.

The disclosed technology exploits an alternative approach to use the biological toxins and peptides that act in the hemocoel for insect pest control, targeting the needs for environmentally friendly and economical methods for reducing damage to both crop and ornamental plants from insect infestation.

### Begomoviruses

Begomoviruses, viruses in the genus Begomovirus (Geminiviridae), are plant viruses transmitted in a persistent, circulative manner by whiteflies in the B. tabaci species complex (Rosen et al., 2015) with genomes comprised of one or two molecules of 2.5 to 2.8 kb circular single stranded DNA. The genus Begomovirus is the largest genus of viruses, with more than 600 species or strains (Briddon et al., 2010).

Remarkably, the whitefly B. tabaci is the sole vector for all begomoviruses, with B. tabaci MEAM1 capable of transmitting viruses to and from numerous host plants. The disclosure provides a fusion protein comprising the TYLCV coat or capsid protein (CP). TYLCV is a monopartite Begomovirus and a major pathogen of tomato in tropical and subtropical regions throughout the world, and is also a pathogen of eggplant, potato, tobacco, bean and pepper (Glick et al., 2009).

Begomovirus transmission by B. tabaci is circulative and persistent, requiring an average latent period of 4 to 12 hr prior to virus transmission (Cohen et al., 1989; Rosell et al., 1999). The virus can readily be detected in the hemolymph after 2 hr of acquisition (Rosell et al., 1999).

Following ingestion of phloem by the whitefly vector, begomoviruses cross the gut epithelium, enter the hemocoel and then the primary salivary gland (PSG) for delivery with saliva during subsequent feeding for transmission to occur. The Begomovirus TYLCV is detected in the stylets, food canal, midgut, hemolymph and salivary canal of B. tabaci fed on virus-infected plants. Virus binding (mediated by the capsid protein) to receptors on the epithelia of the gut or PSG determines transmission specificity, i.e., determines why begomoviruses are only transmitted by B. tabaci. While there is variation between whitefly species (Polston et al., 2014), a fusion protein based on the CP of TYLCV would cross the gut epithelium of all Bemisia-, but no non-Bemisia-, whiteflies.

### Fusion Proteins

In one aspect, described herein is a fusion protein comprising a carrier protein attached to a second peptide via a peptide linker, wherein the carrier protein is derived from a tomato yellow leaf curl virus (TYLCV). In some embodiments, the carrier protein is a TYLCV coat protein (CP).

The coat protein (CP) of a whitefly-vectored plant virus, Tomato yellow leaf curl virus (TYLCV: Begomovirus), may provide that delivery system to allow for exploitation of this and similar toxins that act within the hemocoel for whitefly control. It is contemplated that fusion proteins comprising a TYLCV coat protein, a linker (e.g., a proline-rich linker (P)) and an insect-specific neurotoxin or physiological disruptor would allow for the development of transgenic resistance to the whitefly.

Exemplary physiological disruptors include allatostatin, chitinase or diuretic hormone, metabolitic or analogue thereof. In some embodiments, the physiological disruptor is Manduca sexta allatostatin (Manse-AS); cockroach allatostatin such as those found in either of the following species Diplotera punctata or Periplaneta americana or blowfly allatostatin such as in the species Calliphora vomitaria. Alternatively insect specific enzymes can be used such as an insect chitinase for example, those found in M. sexta; Bombyx mori, the mosquito Anopheles gambiae; fall webworm Hyphantria cunea; beetle Phaedon cochleariae; or Lacanobia oleracea.

In some embodiments, the physiological disruptor is an allatostatin such as Manduca sexta allatostatin, Diploptera punctata allatostatin, Periplaneta americana allatostatin, or Calliphora vomitaria allatostatin. In some embodiments, the physiological disruptor is an insect chitinase such as M. sexta chitinase, Bombyx mori chitinase, Anopheles gambiae chitinase, Hyphantria cunea chitinase, Phaedon cochleariae chitinase, or Lacanobia oleracea chitinase. In some embodiments, the physiological disruptor is aninsect diuretic hormone such as that isolated from M. sexta.

In some embodiments, the coat protein comprises an amino acid sequence that is at least 85% identical to the amino acid sequence set forth in SEQ ID NO: 2. In some embodiments, the coat protein comprises an amino acid sequence that is at least 85%, 86%, 87%, 88%, 89%, 90%, 91%, 92%, 93%, 94%, 95%, 96%, 97%, 98%, 99% or more identical to SEQ ID NO: 2. In some embodiments, the coat protein comprises an amino acid sequence set forth in SEQ ID NO: 2.

Nucleotide sequences encoding the coat protein are also contemplated. In some embodiments, the nucleotide sequence encoding the coat protein comprises a sequence that is at least 85% identical to the nucleotide sequence set forth in SEQ ID NO: 1. In some embodiments, the nucleotide sequence encoding the coat protein comprises a nucleotide acid sequence that is at least 85%, 86%, 87%, 88%, 89%, 90%, 91%, 92%, 93%, 94%, 95%, 96%, 97%, 98%, 99% or more identical to SEQ ID NO: 1. In some embodiments, the nucleotide sequence encoding the coat protein comprises the sequence set forth in SEQ ID NO: 1.

The term “linker” as used herein refers to short peptide sequences that occur between functional protein domains and link the functional domains together. Linkers are generally classified into three categories according to their structures: flexible linkers, rigid linkers, and in vivo cleavable linkers. A flexible linker is often composed of flexible residues like glycine and serine so that the adjacent protein domains are free to move relative to one another. A linker also may play a role in releasing the free functional domain in vivo (as in in vivo cleavable linkers). Linkers may offer many other advantages for the production of fusion proteins, such as improving biological activity, increasing expression yield, and achieving desirable pharmacokinetic profiles. A linker is generally from about 3 to about 15 amino acids long, in some embodiments about 5 to about 10 amino acids long; however, longer or shorter linkers may be used or the linker may be dispensed with entirely. In some embodiments, the linker is a proline-rich linker or a glycine linker. In some embodiments, the linker is a proline-rich linker. In some embodiments, the proline-rich linker comprises the amino acid sequence set forth in SEQ ID NO: 4. In some embodiments, the proline-rich linker is encoded by the nucleotide sequence set forth in SEQ ID NO: 3.

In some embodiments, the second peptide is an insect-specific toxin. Insect-specific toxins such as those derived from Bacillus thuringiensis have been employed successfully for the management of numerous arthropod pests (Sanahuja et al., Plant Biotech J, 9:283-300, 2011). However, a diverse range of insect-specific insecticidal peptides and neurotoxins that act within the insect hemocoel remain an untapped resource for pest management efforts. Insect-specific toxins either act at the gut epithelium or act within the hemocoel (body cavity), requiring appropriate delivery to their site of action (Windley et al., Toxins Basel, 4:191-277, 2012). Due to the challenge of appropriate delivery to their target site, hemocoelic toxins have not been widely adopted.

The venom from a wide range of predatory species (e.g., scorpions, wasps, predaceous mites, cone snails, anemones, lacewings, and parasitoids) provides an outstanding resource for isolation of insect-specific neurotoxins. In addition, insect-specific toxins are produced by Xenorhabdus and Photorhabdus bacteria that reside in entomopathogenic nematodes. These insecticidal neurotoxins typically target sodium, potassium, calcium, or chloride channels. With few exceptions, these neurotoxins are not orally active and require appropriate delivery systems to access their target site, the nerves.

Arachnids have a venom apparatus and secrete a complex chemical mixture of low molecular mass organic molecules, enzymes and polypeptide neurotoxins designed to paralyze or kill their prey. Based on the number of species and number of toxins present in the venoms of those examined, there are an estimated 0.5-1.5 million arachnid-derived insecticidal peptides. It is predicted that there are at least 10 million bioactive spider-venom peptides. ArachnoServer is a database containing information on the sequence, three-dimensional structure, and biological activity of protein toxins derived from spider venom. Of 800 peptides in the ArachnoServer 2.0 Database, 136 are insecticidal peptides, of which 38 are insect selective, 34 are nonselective, and 64 are unknown phyletic selectivity.

Arthropod-derived neuropeptides, enzymes, and hormones that function to regulate insect development and maintain homeostasis (e.g., diuretic hormones and juvenile hormone esterase) also include peptides that have potentially insecticidal effects when delivered outside their normal physiological timeframe. Although these peptides act as endogenous regulators and provide insect specificity, a major drawback of using these peptides as insecticides is that high concentrations of these peptides are usually required to overcome natural regulatory mechanisms that restore appropriate physiological levels within the insect.

A few arthropod-derived insecticidal peptides (e.g., proctolin and Aedes aegypti trypsin modulating oostatic factor (TMOF)) are transported at low levels across the insect gut epithelium. The target specificity of these naturally occurring arthropod-derived insecticidal peptides is particularly appealing for the development of novel pest management technologies where appropriate delivery systems are provided.

Spider, Hadronyche versuta omega atracotoxin, Hv1a is an insecticidal peptide derived from the venom of an Australian funnel-web spider (Hadronyche versuta), specifically inhibits insect but not mammalian voltage-gated calcium channels. Hvla is highly toxic by injection towards many different insect pests including species from the orders Lepidoptera, Coleoptera, Diptera, and Dictyoptera, and is ineffective after oral ingestion. However, Hv1a is orally toxic against one tick species (Amblyomma americanum), which may be related to differences in gut physiology associated with blood feeding. The spider-derived toxins, such as Hv1a, that contain a disulfide pseudoknot are classified as inhibitor cysteine-knot (ICK) motif toxins. The cysteine-knot in these neurotoxins results in strong chemical, thermal, and biological stability, contributing to their persistence and making them particularly attractive for use as model toxins.

In some embodiments, the insect specific toxin is an insect specific neurotoxin. In some embodiments, the insect specific neurotoxin comprises an arthropod-derived neuropeptide, enzyme, and/or hormone. In some embodiments, the arthropod-derived insecticidal peptide is Hv1a (nucleotide sequence set forth in SEQ ID NO: 7; amino acid sequence set forth in SEQ ID NO: 8), proctolin, or Aedes aegypti trypsin modulating oostatic factor. Insecticidal activity of exemplary toxins and peptides are provided below in Table 1.

| Source | Toxin/Peptide | Targets | References |
|---|---|---|---|
| Bacteria | Makes Caterpillars | Cell apoptosis | Ffrench-Constant |
| | Floppytoxin 1(Mcf1) | | et al (2007) |
| | Photorhabdusvirulence | Unknown | Ffrench-Constant |
| | cassettes | | et al (2007) |
| Arachnids | Hv1a | Ion channels | Windley et al |
| | Pl1a | Cell membrane | (2012) |
| | (Predicted 0.5-1.5 million | Neurons | |
| | insecticidal peptides) | | |
| Vertebrates | Cobra neurotoxins | Muscles | Primor et al. (1980) |
| | | Axonal conduction | |
| | | Hemolytic activity | |
| Insects | Adipokinetic hormone | Metabolic functions | Stone et al (1976) |
| | | Locomotion | |
| | Diuretic hormone | Water balance | Gade (2004) |
| | | Feeding behavior | |
| | Cockroach, Cricket, Moth | JH biosynthesis | Gade (2004), |
| | Allatostatin | (Crickets), Ecdysteroids | Schoofs et al. |
| | | (Lepidoptera) | (1998) |

### Vectors

The disclosure further provides a vector comprising one or more nucleotide sequences described herein (e.g., a nucleotide sequence set forth in SEQ ID NO: 1, 3, 5, 6, 7, 9, and/or 11).

The term “vector” as used herein encompasses (but is not limited to) a phage, plasmid, viral or retroviral vector, as well as artificial chromosomes, such as bacterial or yeast artificial chromosomes. Moreover, the term also relates to targeting constructs which allow for random or site-directed integration of the targeting construct into genomic DNA. Such target constructs, preferably, comprise DNA of sufficient length for either homologous or heterologous recombination as described in detail below. The vector comprising the polynucleotides of described herein may comprise selectable markers for propagation and/or selection in a host. Further, the vector may be prepared from native (endogenous) and/or foreign (exogenous, heterologous) sequences with respect to the host.

Preferably, the vector referred to herein is suitable as a cloning vector, i.e., replicable in microbial systems. Such vectors ensure efficient cloning in bacteria, yeasts or fungi and make possible the stable transformation of plants. Examples include, e.g., various binary and co-integrated vector systems which are suitable for T DNA-mediated transformation. Such vector systems are generally characterized in that they contain at least the vir genes, which are required for the Agrobacterium-mediated transformation, and the sequences which delimit the T-DNA (T-DNA border). These vector systems also optionally comprise further cis-regulatory regions such as promoters and terminators and/or selection markers with which suitable transformed host cells or organisms can be identified. While co-integrated vector systems have vir genes and T DNA sequences arranged on the same vector, binary systems are based on at least two vectors, one of which bears vir genes, but no T-DNA, while a second one bears T DNA, but no vir gene. As a consequence, the last-mentioned vectors are relatively small, easy to manipulate and can be replicated both in E. coli and in Agrobacterium. An overview of binary vectors and their use can be found in Hellens et al, Trends in Plant Science (2000) 5, 446-451. Furthermore, by using appropriate cloning vectors, an expression cassette can be introduced into host cells or organisms such as plants or animals and, thus, be used in the transformation of plants, such as those which are published, and cited, in: Plant Molecular Biology and Biotechnology (CRC Press, Boca Raton, Florida), chapter 6/7, pp. 71-119 (1993); F. F. White, Vectors for Gene Transfer in Higher Plants; in: Transgenic Plants, vol. 1, Engineering and Utilization, Ed.: Kung and R. Wu, Academic Press, 1993, 15-38; B. Jenes et al., Techniques for Gene Transfer, in: Transgenic Plants, vol. 1, Engineering and Utilization, Ed.: Kung and R. Wu, Academic Press (1993), 128-143; Potrykus, Annu. Rev. Plant Physiol. Plant Molec. Biol. 42 (1991), 205 225.

Suitable vector backbones are, in some embodiments, derived from vectors known in the art such as Okayama-Berg cDNA expression vector pcDV1 (Pharmacia), pCDM8, pRc/CMV, pcDNA1, pcDNA3 (Invitrogene) or pSPORT1 (GIBCO BRL). Further examples of typical fusion expression vectors are pGEX (Pharmacia Biotech Inc; Smith, D. B., and Johnson, K. S. (1988) Gene 67:31-40), pMAL (New England Biolabs, Beverly, MA) and pRIT5 (Pharmacia, Piscataway, NJ), where glutathione S-transferase (GST), maltose E-binding protein and protein A, respectively, are fused with the nucleic acid of interest encoding a protein to be expressed.

In some embodiments, the vector comprising one or more nucleotide sequences described herein is propagated and amplified in a plant cell. In some embodiments, one copy of the vector is propagated and amplified in a plant cell. In some embodiments, two or more (e.g., 3, 4, 5, 6 7, 8 or more) copies of the vector are propagated and amplified in a plant cell.

In some embodiments, the vector described herein comprises a promoter (e.g., a phloem-specific promoter, a leaf-specific promoter, a light activated promoter or a leaf-damage activated promoter) operably linked to a nucleotide sequence described herein. In some embodiments, the nucleotide sequence is further operably linked to termination signals and/or other regulatory elements.

The term “promoter” as used herein refers to a nucleotide sequence, usually upstream (5′) to its coding sequence, which controls the expression of the coding sequence by providing the recognition site for RNA polymerase and other factors required for proper transcription. “Promoter” includes a minimal promoter that is a short DNA sequence comprised, in some cases, of a TATA box and other sequences that serve to specify the site of transcription initiation, to which regulatory elements are added for enhancement of expression. “Promoter” also refers to a nucleotide sequence that includes a minimal promoter plus regulatory elements and that is capable of controlling the expression of a coding sequence or functional RNA. This type of promoter sequence consists of proximal and more distal upstream elements, the latter elements often referred to as enhancers. Accordingly, an “enhancer” is a DNA sequence, which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements, derived from different promoters found in nature, or even be comprised of synthetic DNA segments.

The terms “operably linked” or “functionally linked” refer to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

The term “constitutive promoter” as used herein refers to a promoter that is able to express the open reading frame (ORF) in all or nearly all of the plant tissues during all or nearly all developmental stages of the plant. Each of the transcription-activating elements do not exhibit an absolute tissue-specificity, but mediate transcriptional activation in most plant tissues at a level of at least 1% reached in the plant tissue in which transcription is most active. “Constitutive expression” refers to expression using a constitutive promoter.

In some embodiments, an operable linkage comprises a sequential arrangement of a nucleotide sequence encoding a promoter, with a nucleic acid sequence to be expressed, and optionally, additional regulatory elements such as, for example, polyadenylation or transcription termination elements, enhancers, introns, etc, such that the nucleotide sequence of interest is expressed under the appropriate conditions (i.e., in a plant cell). Suitable arrangements include, e.g., those in which the nucleic acid sequence to be expressed is placed downstream (i.e., in 3′-direction) of the transcription regulating nucleotide sequence such that both sequences are covalently linked. Optionally, additional sequences may be inserted in-between the two sequences. Such sequences may be, for example, linker or multiple cloning sites. Furthermore, sequences can be inserted which encode parts of a fusion protein, in the event that a fusion protein comprising the product of the nucleic acid disclosed herein is desired. Preferably, the distance between the polynucleotide to be expressed and the transcription regulating nucleotide sequence is not more than 200 base pairs, such as not more than 100 base pairs or not more than 50 base pairs.

### Expression in a Host Cell

In another aspect, described herein is a method for expressing a polynucleotide of interest in a host cell comprising introducing an expression cassette or vector described herein into the host cell and expressing the polynucleotide of interest in the host cell. Methods of editing the genome of a plant cell are also provided.

The term “expression” as used herein refers to the transcription and/or translation of an endogenous gene, ORF or portion thereof, or a transgene in plants. For example, in the case of antisense constructs, expression may refer to the transcription of the antisense DNA only. In addition, expression refers to the transcription and stable accumulation of sense (mRNA) or functional RNA. Expression may also refer to the production of protein.

The “expression pattern” of a promoter (with or without enhancer) is the pattern of expression levels, which shows where in the plant and in what developmental stage transcription is initiated by said promoter. Expression patterns of a set of promoters are said to be complementary when the expression pattern of one promoter shows little overlap with the expression pattern of the other promoter. The level of expression of a promoter can be determined by measuring the steady state concentration of a standard transcribed reporter mRNA. This measurement is indirect since the concentration of the reporter mRNA is dependent not only on its synthesis rate, but also on the rate with which the mRNA is degraded. Therefore, the steady state level is the product of synthesis rates and degradation rates. When promoters are compared in this way, techniques available to those skilled in the art are hybridization S1-RNAse analysis, northern blots and competitive RT-PCR. This list of techniques in no way represents all available techniques, but rather describes commonly used procedures used to analyze transcription activity and expression levels of mRNA. The analysis of transcription start points in practically all promoters has revealed that there is usually no single base at which transcription starts, but rather a more or less clustered set of initiation sites, each of which accounts for some start points of the mRNA. Since this distribution varies from promoter to promoter the sequences of the reporter mRNA in each of the populations would differ from each other. Since each mRNA species is more or less prone to degradation, no single degradation rate can be expected for different reporter mRNAs. It has been shown for various eukaryotic promoter sequences that the sequence surrounding the initiation site (“initiator”) plays an important role in determining the level of RNA expression directed by that specific promoter. This includes also part of the transcribed sequences. The direct fusion of promoter to reporter sequences would therefore lead to suboptimal levels of transcription. A commonly used procedure to analyze expression patterns and levels is through determination of the ‘steady state’ level of protein accumulation in a cell. Commonly used candidates for the reporter gene, known to those skilled in the art are beta-glucuronidase (GUS), chloramphenicol acetyl transferase (CAT) and proteins with fluorescent properties, such as green fluorescent protein (GFP) from Aequora victoria. In principle, however, many more proteins are suitable for this purpose, provided the protein does not interfere with essential plant functions. For quantification and determination of localization a number of tools are suited. Detection systems can readily be created or are available which are based on, e.g., immunochemical, enzymatic, fluorescent detection and quantification. Protein levels can be determined in plant tissue extracts or in intact tissue using in situ analysis of protein expression. Generally, individual transformed lines with one chimeric promoter reporter construct may vary in their levels of expression of the reporter gene. Also frequently observed is the phenomenon that such transformants do not express any detectable product (RNA or protein). The variability in expression is commonly ascribed to position effects, although the molecular mechanisms underlying this inactivity are usually not clear.

The expression of a polynucleotide of interest can be determined by various well known techniques, e.g., by Northern Blot or in situ hybridization techniques as described in WO 02/102970. The term “expression” as used herein refers to the transcription and/or translation of nucleic acid (e.g., transgene) in a plant cell. The expression of a polynucleotide of interest in a host cell (e.g., a plant cell) can be determined by various well known techniques, e.g., by Northern Blot or in situ hybridization techniques as described in WO 02/102970, the disclosure of which is incorporated by reference in its entirety.

In some embodiments, the host cell is from a plant (e.g., a plant cell, a plant seed or other plant part). To confirm the presence of the transferred polynucleotide of interest in transgenic cells and plants, a variety of assays may be performed. The expression of a polynucleotide of interest in a host cell (e.g., a plant cell) can be determined by various well known techniques, e.g., by Northern Blot or in situ hybridization techniques as described in WO 02/102970, the disclosure of which is incorporated by reference in its entirety. Such assays include, Northern Blot or in situ hybridization techniques as described in WO 02/102970, in situ hybridization and nucleic acid-based amplification methods such as PCR or RT-PCR or TaqMan; immunological assays such as ELISAs and Western blots, and also, by analyzing the phenotype of the whole regenerated plant.

### Transgenic Plants

A transgenic plant or plant part comprising a vector (or nucleotide sequence) described herein is specifically contemplated.

The term “plant” as used herein refers to a photosynthetic, eukaryotic multicellular organism. The term “plant” encompasses whole plants, ancestors and progeny of the plants, wherein each of the aforementioned comprise the gene/nucleic acid of interest. The term “plant parts” as used herein encompasses all components of a plant including seeds, shoots, stems, leaves, roots, flowers, plant tissues, plant organs, plant cells, suspension cultures, callus tissue, embryos, meristematic regions, gametophytes, sporophytes, pollen, microspores and propagules. A “propagule” is any kind of organ, tissue, or cell of a plant capable of developing into a complete plant. A propagule can be based on vegetative reproduction (also known as vegetative propagation, vegetative multiplication, or vegetative cloning) or sexual reproduction. A propagule can therefore be seeds or parts of the non-reproductive organs, like stem or leaf. In particular, with respect to Poaceae, suitable propagules can also be sections of the stem, i.e., stem cuttings.

In some embodiments, the plant is a monocotyledonous plant, or the plant part is derived from a monocotyledonous plant. In some embodiments, the plant is a dicotyledonous plant, or the plant part is derived from a dicotyledonous plant.

A transgenic plant or plant part comprising a vector described herein is specifically contemplated. The vector may be present in the cytoplasm of the plant or may be incorporated into the genome either heterologous or by homologous recombination.

In some embodiments, the plant (or plant part) is derived from the genera: Ananas, Musa, Vitis, Fragaria, Lotus, Medicago, Onobrychis, Trifolium, Trigonella, Vigna, Citrus, Carica, Persea, Prunus, Syragrus, Theobroma, Coffea, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Mangifera, Cichorium, Helianthus, Lactuca, Bromus, Asparagus, Antirrhinum, Heterocallis, Nemesia, Pelargonium, Panicum, Pennisetum, Ranunculus, Senecio, Salpiglossis, Cucurbita, Cucumis, Browaalia, Lolium, Malus, Apium, Gossypium, Vicia, Lathyrus, Lupinus, Pachyrhizus, Wisteria, Stizolobium, Agrostis, Phleum, Dactylis, Sorghum, Setaria, Zea, Oryza, Triticum, Secale, Avena, Hordeum, Saccharum, Poa, Festuca, Stenotaphrum, Cynodon, Coix, Olyreae, Phareae, Glycine, Pisum, Psidium, Passiflora, Cicer, Phaseolus, Lens, or Arachis.

In some embodiments, the plant (or plant part) is from the family of Poaceae, such as the genera Hordeum, Secale, Avena, Sorghum, Andropogon, Holcus, Panicum, Oryza, Zea, Triticum, for example the genera and species Hordeum vulgare, Hordeum jubatum, Hordeum murinum, Hordeum secalinum, Hordeum distichon, Hordeum aegiceras, Hordeum hexastichon, Hordeum hexastichum, Hordeum irregulare, Hordeum sativum, Hordeum secalinum, Secale cereale, Avena sativa, Avena fatua, Avena byzantina, Avena fatua var. sativa, Avena hybrida, Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cernuum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum, Panicum militaceum, Oryza sativa, Oryza latifolia, Zea mays, Triticum aestivum, Triticum durum, Triticum turgidum, Triticum hybernum, Triticum macha, Triticum sativum or Triticum vulgare.

In some embodiments, the plant is a squash plant, a watermelon plant, a melon plant, a bean plant, a tomato plant, a potato plant, a cotton plant, an okra plant or a pepper plant. In some embodiments, the plant is a cucurbit plant.

### Methods of Producing a Transgenic Plant

The disclosure also provides a method for producing a transgenic plant comprising introducing one or more nucleotide sequences described herein (e.g., a nucleotide sequence at least 85% identical to SEQ ID NO: 11) into the genome of the plant. In some embodiments, the nucleotide sequence is operably linked to a phloem-specific promoter, leaf-specific promoter, a light activated promoter or a leaf-damage activated promoter. In some embodiments, the promoter is a constitutive promoter.

A variety of techniques are available and known to those skilled in the art for introduction of constructs (e.g., vectors) into a host cell (e.g., plant cell). Exemplary techniques include transformation with DNA employing A. tumefaciens or A. rhizogenes as the transforming agent, liposomes, PEG precipitation, electroporation, DNA injection, direct DNA uptake, microprojectile bombardment, particle acceleration, CRISPR and the like (see, for example, EP 295959 and EP 138341). However, cells other than plant cells may be transformed with the vector described herein. The general descriptions of plant expression vectors and reporter genes, and Agrobacterium and Agrobacterium-mediated gene transfer, can be found in Gruber et al. (1993).

Other transformation methods are available to those skilled in the art, such as direct uptake of foreign DNA constructs (see, e.g., EP 295959), techniques of electroporation (Fromm 1986) or high velocity ballistic bombardment with metal particles coated with the nucleic acid constructs (e.g., U.S. Pat. No. 4,945,050). Once transformed, the cells can be regenerated by those skilled in the art. Those skilled in the art will appreciate that the choice of method might depend on the type of plant, i.e., monocotyledonous or dicotyledonous, targeted for transformation.

In some embodiments, the construct is introduced into a host cell by introducing a genome editing component comprising: a) an enzyme inducing a double-stranded break (DSB) or a nucleic acid encoding same, and optionally a repair nucleic acid molecule, wherein the DSB-inducing enzyme optionally recognizes a predetermined site in the genome of said cell; b) an enzyme inducing a single-stranded break (SSB) or a nucleic acid encoding same, and optionally a repair nucleic acid molecule, wherein the SSB-inducing enzyme optionally recognizes a predetermined site in the genome of said cell; c) a base editor enzyme, optionally fused to a disarmed DSB- or SSB-inducing enzyme, wherein the base editor enzyme preferably recognizes a predetermined site in the genome of said cell; or d) an enzyme effecting DNA methylation, histone acetylation, histone methylation, histone ubiquitination, histone phosphorylation, histone ribosylation or histone citrullination, optionally fused to a disarmed DSB- or SSB-inducing enzyme, wherein the enzyme preferably recognizes a predetermined site in the genome of said cell.

In order to enable a break at a predetermined target site, the enzymes preferably include a binding/recognition domain and a cleavage domain. Particular enzymes capable of inducing double or single-stranded breaks are nucleases or nickases as well as variants thereof, including such molecules no longer comprising a nuclease or nickase function but rather operating as recognition molecules in combination with another enzyme. In recent years, many suitable nucleases, especially tailored endonucleases have been developed comprising meganucleases, zinc finger nucleases, TALE nucleases, Argonaute nucleases, derived, for example, from Natronobacterium gregoryi, and CRISPR nucleases, comprising, for example, Cas9, Cpf1, Csm1, CasX or CasY nucleases as part of the Clustered Regularly Interspaced Short Palindromic Repeats (CRISPR) system. Thus, in some embodiments, the genome engineering component comprises a DSB- or SSB-inducing enzyme or a variant thereof selected from a CRISPR/Cas endonuclease, preferably a CRISPR/Cas9 endonuclease a CRISPR/Cpf1 endonuclease, or a CRISPR/Csm1 endonuclease, a zinc finger nuclease (ZFN), a homing endonuclease, a meganuclease and a TAL effector nuclease.

In some embodiments, the methods described herein comprise introducing one or more vectors comprising the one or more nucleotide sequences into the plant by transformation. In some embodiments, the methods described herein comprise introducing a vector comprising the nucleotide sequence set forth in SEQ ID NO: 10 into the plant by transformation.

If desired, the vector may comprise a selectable marker, which may provide resistance to an antibiotic (e.g., kanamycin, hygromycin or methotrexate) or a herbicide (e.g., phosphinothricin), or a separate vector encoding a selectable marker may be utilized in conjunction with the vector comprising the nucleotide of interest described above. For certain plant species, different antibiotic or herbicide selection markers may be preferred. Selection markers used routinely in transformation include the nptII gene which confers resistance to kanamycin and related antibiotics, the bar gene which confers resistance to the herbicide phosphinothricin, the hph gene which confers resistance to the antibiotic hygromycin, and the dhfr gene which confers resistance to methotrexate.

In some embodiments, a plant virus in the phloem of a plant is engineered to produce the fusion proteins disclosed herein.

Methods for the production and further characterization of stably transformed plants are well-known to the person skilled in the art. As an example, transgenic plant cells are placed in an appropriate selective medium for selection of transgenic cells, which are then grown to callus. Shoots are grown from callus. Plantlets are generated from the shoot by growing in rooting medium. When a selection marker is used, the marker allows for selection of transformed cells as compared to cells lacking the DNA.

In some embodiments, the methods described herein comprise sexually crossing a plant with the transgenic plant described herein.

### Compositions

A composition comprising a fusion protein described herein is also contemplated.

Methods of controlling insect pests comprising contacting an insect with a fusion protein described herein (or a composition described herein, or a transgenic plant described herein), wherein upon ingestion, the fusion protein passes across the gut epithelium and moves into the hemocoel of the insect, thereby killing the insect pest (e.g., whitefly, Bemisia tabaci).

### Methods of Controlling Insect Pests.

The disclosure also provides a method of controlling insect pests, the method comprising feeding an insect with a food source comprising a fusion protein described herein or a composition described herein, wherein upon ingestion, the fusion protein passes across the gut epithelium and moves into the hemocoel of the insect.

In some embodiments, insect gut bacteria synthesize and release the fusion protein into the gut of the insect.

It is to be understood that this invention is not limited to the particular methodology, protocols, cell lines, plant species or genera, constructs, and reagents described as such. It is also to be understood that the terminology used herein is for the purpose of describing particular embodiments only, and is not intended to limit the scope of the present invention, which will be limited only by the appended claims. It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth.

### EXAMPLES

### Materials and Methods

Insects: Bemisia tabaci (Hemiptera: Aleyrodidae) individuals were reared on either Gossypium hirsutum (spring and summer) and Brassica oleracea var. capitata (fall and winter) and kept in Plant Pathology facilities at the University of Florida. Newly emerged adults (1-7 day-old) were used to run the bioassays. All whitefly colonies were maintained in growth chambers at a constant temperature of 26° C. with a 14 h light/10 h dark cycle.

Cloning of TYLCV CP-P-eGFP: The sequence of the cDNA encoding TYLCV coat protein (CP) (SEQ ID NO: 1) in pGEM-TEASY (pGEM-CP) was confirmed. To create the CP-P-eGFP construct, PCR (primers 1 and 2; Table 2) was used to amplify the full CP sequence with addition of a BamHI restriction site at the N-terminus, and 24 nt of the polyproline linker (SEQ ID NO: 3) at the C-terminus. The P-eGFP fragment was amplified from pGEX-JcDNV VP4-P-eGFP (Kemmerer and Bonning, 2018), with primers 3 and 4 (Table 2) adding 24 nt of TYLCV CP at the N-terminus and an EcoRI restriction site at the C-terminus. Fusion PCR with Phusion DNA polymerase (Thermo Fisher), with primers 1 and 4 was used to amplify the entire construct from the two fragments. The full-length cDNA was cloned into pCR-Blunt II-TOPO. TOP10 E. coli were transformed with this plasmid, and PCR with M13F and M13R primers used to identify positive clones, which were confirmed by sequencing. The construct pCR-CP-P-eGFP was digested with BamHI/EcoRI (NEB) and the digested CP-P-eGFP fragment isolated from an agarose gel, cloned into pGEX-4T (GE Healthcare), and confirmed by sequencing using GEX3 and GEX5 universal primers. As a control, P-eGFP, was amplified from pGEX-CP-P-eGFP by PCR with iF primers (Table 2) and cloned into pGEX-4T according to the In-Fusion HD Cloning protocol (Takara Bio). Expression and purification of the recombinant proteins CP-P-eGFP and P-eGFP (FIG. 2) using the pGEX-4T system is under way.

TABLE 2
Primer sequences used for cloning of CP-P-EGFP andPp-eGFP

| Primer Name | Sequence |
|---|---|
| 1 (SEQ ID NO: 13) | ATAGGATCCTATATGTCGAAGCGACC |
| 2 (SEQ ID NO: 14) | TGGTGACGGGGGAGCGTCGTCCCCATTTGATATTGAATCATAGA |
| 3 (SEQ ID NO: 15) | CTATTTCTATGATTCAATATCAAATGGGGACGACGCTCCCCCGT |
| 4 (SEQ ID NO: 16) | CGCGAATTCGCGTTAGCCGCTTTACTT |
| P-EGFP IF 1 (SEQ ID NO: 17) | GGTTCCGCGTGGATCCACTGGGGACGACGCTCCCCCG |
| P-EGFP IR 1 (SEQ ID NO: 18) | GATGCGGCCGCTCGAGACTTTAGCCGCTTTACTTGTACAG |
| | CTCGTCCAT |

### Expression and Purification of TYLCV CP-P-mCherry:

The construct pGEX 4T-CP-P-eGFP was digested with BamHI/EcoRI (NEB) and the digested CP-P fragment was amplified using primers 5 and 6 (Table 2). The mCherry was amplified from a peptide X-mCherry fragment using primers 7 and 8 (Table 2). Both TYLCV CP-P and mCherry fragments were assembled by fusion PCR using primers 9 and 10 (Table 2).

TABLE 2
Primer sequences used for cloning of TYLCV CP-P-mCherry

| Primer Name | Sequence |
|---|---|
| 5 (SEQ ID NO: 19) | TTAGAATTCCTTTCGAAGCGACCAGGCGATATAATCA |
| 6 (SEQ ID NO: 20) | GTTATCCTCCTCGCCCTTGCTCACTCCTACGGGAGTGGGACTTG |
| 7 (SEQ ID NO: 21) | CAAGTCCCACTCCCGTAGGAGTGAGCAAGGGCGAGGAGGATAAC |
| 8 (SEQ ID NO: 22) | ATAAGCATAAAGCTTATTTTACTTGTACAGCTCGTCCATGCCGC |
| 9 (SEQ ID NO: 23) | TTAGAATTCCTTTCGAAGCG |
| 10 (SEQ ID NO: 24) | ATAAGCATAAAGCTTATTTTACTTGTA |

The full-length TYLCV CP-P-mCherry cDNA was cloned into pFastBac HT-A (Invitrogen), later transformed in E. coli TOP 10 and confirmed by sequencing using pFastBac universal primers. Confirmed constructs were cloned into DH10Bac cells and transformed bacmid was confirmed bu running a PCR using pUC/M13 universal primers. Finally, Sf9 cells were transfected with the vTYLCV CP-P-mCherry. Sf9 cell monolayer cultures were maintained in Sf900 SFMIII growth medium (Life Technologies/Thermo Fisher Scientific, Carlsbad, CA) at 27° C. Sf9 cells were transfected with the pFastBac HT-A/TYLCV CP-PmCherry (500 ng). The resulting recombinant baculovirus (P1) was further amplified until obtaining P3 with an appropriate virus titer. Titering of the several baculoviral stocks was performed by plaque assay according to the Bac-to-Bac Manual (Invitrogen, Carlsbad, CA). For large scale protein expression, Sf9 cells were infected at a multiplicity of infection (MOI) of 5. Cells were harvested 96 hours post-infection and stored at −20° C. until use.

TYLCV CP-P-mCherry protein was purified by affinity chromatography batch method (ThermoFisher). After 96 h of baculovirus infection, Sf9 cells were collected and later solubilized in cell lysis buffer [50 mM Tris, IGEPAL at critical micelle concentration-CMC (0.08 mM); 1 mM PMSF; pH 7.4]. Later, Sf9 cells were sonicated (10 s sonication/20 s rest, for 2 min) and samples were placed in a tube rotator overnight at 4° C. Next day, the lysate was centrifuged at 12,000 rpm for 20 min a 4° C. in Beckman J2-21 centrifuge. The supernatant was diluted 1:1 in equilibration buffer (50 mM Tris, 150 mM NaCl, 10 mM Imidazole; pH 7.4). Two-resin bed volumes of protein aliquot was mixed with Ni-NTA agarose (Sigma-Aldrich, St Louis, MO). Resin was first equilibrated, and sample was later added and incubate for 30 min on an end-over-end rotator at 4° C. Resin was then washed with the same buffer but containing an increased amount of Imidazole (20 mM). Finally, protein was eluted with 1 mL ml elution buffer containing 250 mM imidazole for 15 min on end-over-end rotator at 4° C. The protein was concentrated and dialyzed against PBS Buffer (pH 7.4). The protein concentration of the solubilized fraction was determined by Bradford assay (BioRad, Hercules, CA), further analyzed in a 10% SDS PAGE gel and western blot using mCherry antibodies (dilution 1:5,000).

Expression and purification of P-mCherry and mCherry proteins: The PCR-amplified P-mCherry and mCherry fragments were cloned into pBAD His B (Invitrogen). TOP 10 E. coli cells were transformed either with the pBAD His B/P-mCherry or pBAD His B/mCherry plasmids using heat-shock standard protocol (Invitrogen), and positive colonies were checked by colony PCR using pBAD primers and restriction digestion. Protein expression in E. coli cells was induced using 0.002% L-Arabinose for 16 h in a shaker at 37° C., 230 rpm.

Both P-mCherry and mCherry were purified by affinity chromatography batch method (ThermoFisher). In this case, pelleted cells were resuspended in 50 mM Tris, 1% IGEPAL; 1 mM PMSF; pH 7.4, and lysed for 30 min on an end-over-end rotator at 4° C. Further purification steps using Ni-NTA agarose, proteins concentration and buffer dialysis were conducted as indicated previously for the TYCV CP-P-mCherry protein.

Trypan Blue injections in B. tabaci adults: To visualize the pericardial cells and dorsal aorta with the whitefly body, we injected whiteflies with 10 nl Trypan blue using a microinjector as describes previously (Fukatsu et al., 2001). After 1 hour, whiteflies were washed in 70% ethanol to remove wax from body and wings and later observed through a Zeiss microscope.

### Example 1—Ability of TYLCV to Deliver a Reporting Protein into the Whitefly Hemoceoel

Baculovirus or bacterial expressed and further purified recombinant proteins (HismCherry, HisP-mCherry and HisCP-P-mCherry) were used to fed whiteflies (30 individuals per each technical replicate) using membrane feeding assay. Whiteflies were collected and caged inside a feeding chamber with two parafilm membranes at the top of it sandwiching preparations of the recombinant proteins in 20% sucrose. Control whiteflies were fed on PBS buffer containing 20% sucrose. Whiteflies were examined for fluorescence after 16 h using a Zeiss Axioplan 2 fluorescence microscope, with whitefly wings previously removed to avoid interference. Four biological replicates were conducted for the four treatments, with a total number of 120 whiteflies examined per treatment.

In order to check if TYLCV coat protein was able to cross the whitefly gut barriers, whiteflies were fed on TYLCV CP-P-mCherry. P-mCherry and mCherry proteins were used as control. Whiteflies were placed inside feeding cages and allowed to acquire the protein from a parafilm membrane for an overnight period (16 h). After feeding time, individuals were paralyzed using ice and later introduced in 70% ethanol to remove the wax lawyer covering the whole body and also the wings were severed so a clear view of the internal organs was achieved. PBS with 20% sucrose was also introduced as an additional control to check the background mCherry florescence of the whitefly organs. Whitefly bodies were individually observed using a fluorescence microscope for the presence of mCherry. In the treatment with whiteflies fed on TYLCV CP-P-mCherry, mCherry fluorescence was detected in the midgut region (most of the times, only partially) and also in pericardial cells/dorsal aorta (data now shown). In control treatments (P-mCherry and mCherry), mCherry fluorescence was limited to the gut area. Little mCherry background was observed when whiteflies feed on PBS control diet. These results indicated that TYLCV CP fusion protein was successfully recognized by specific receptors in the brush membrane, thus crossing the gut barriers into the whitefly hemocoel and thus successfully delivering mCherry.

### Example 2-Toxicity of CP-Toxin Fusion Proteins in Membrane Feeding Assays

Next, delivery of the insect-specific hexatoxin Hv1a neurotoxin using TYLCV CP fusion proteins was tested.

TYLCV CP-P-Hv1a and TYLCV CP-P-Hv1 am proteins will be purified by affinity chromatography batch method (ThermoFisher), same as described for the purification of the TYLCV CP-P-mCherry protein. After 96 h of baculovirus infection, Sf9 cells will be collected and later solubilized in cell lysis buffer [50 mM Tris, IGEPAL at critical micelle concentration—CMC (0.08 mM); 1 mM PMSF; pH 7.4]. Later, Sf9 cells will be sonicated (10 s sonication/20 s rest, for 2 min) and samples were placed in a tube rotator overnight at 4° C. The next day, the lysate will be centrifuged at 12,000 rpm for 20 min a 4° C. in Beckman J2-21 centrifuge. The supernatant will be diluted 1:1 in equilibration buffer (50 mM Tris, 150 mM NaCl, 10 mM Imidazole; pH 7.4). Two-resin bed volumes of protein aliquot will be mixed with Ni-NTA agarose (Sigma-Aldrich, St Louis, MO). Resin will be first equilibrated, and sample later added and incubated for 30 min on an end-over-end rotator at 4° C. Resin will be then washed with the same buffer but containing an increased amount of Imidazole (20 mM). Finally, protein will be eluted with 1 mL elution buffer containing 250 mM imidazole for 15 min on end-over-end rotator at 4° C. The protein will be concentrated and dialyzed against PBS Buffer (pH 7.4). The protein concentration of the solubilized fraction will be determined by Bradford assay (BioRad, Hercules, CA) and further analyzed in a 10% SDS PAGE gel and western blot using TYLCV CP antibodies (dilution 1:500).

It is contemplated that the data will demonstrate 1) that TYLCV CP can deliver heterologous molecules into the whitefly hemocoel, and 2) the toxicity of a CP-toxin fusion to whiteflies in membrane feeding assays. This proof of concept work will provide the basis for testing different systems for delivery of CP-toxin fusions for crop protection. These systems could include the use of transgenic plants (including trap plants), or paratransgenic approaches (i.e., the use of avirulent plant virus vectors or insect gut-residing bacteria) for delivery of the CP-toxin fusion protein into the phloem.

### REFERENCES

Aragao, F. J. L., Faria, J. C., 2009. First transgenic geminivirus-resistant plant in the field. Nature Biotechnology 27, 1086-1088.
Atkinson, R. K., Vonarx, E. J., Howden, M. E. H., 1996. Effects of whole venom and venom fractions from several Australian spiders, including Atrax (Hadronyche) species, when injected into insects. Comp Biochem Phys C 114, 113-117.
Bonning, B. C., Chougule, N. P., 2014. Delivery of intrahemocoelic peptides for insect pest management. Trends Biotechnol 32, 91-98.
Bonning, B. C., Pal, N., Liu, S., Wang, Z., Sivakumar, S., Dixon, P. M., King, G. F., Miller, W. A., 2014. Toxin delivery by the coat protein of an aphid-vectored plant virus provides plant resistance to aphids. Nat Biotechnol 32, 102-105.
Briddon, R. W., Markham, P. G., 2000. Cotton leaf curl virus disease. Virus Research 71, 151-159.
Briddon, R. W., Patil, B. L., Bagewadi, B., Nawaz-ul-Rehman, M. S., Fauquet, C. M., 2010. Distinct evolutionary histories of the DNA-A and DNA-B components of bipartite begomoviruses. BMC Evolutionary Biology 10:97.
Castle, S. J., Palumbo, J. C., Prabhaker, N., Horowitz, A. R., Denholm, I., 2010. Ecological determinants of Bemisia tabaci resistance to insecticides, In: Stansly, P. A.,
Naranjo, S. E. (Eds.), Bemisia: bionomics and management of a global pest. Springer, Dordrecht.
Chong, Y., Hayes, J. L., Sollod, B., Wen, S., Wilson, D. T., Hains, P. G., Hodgson, W. C., Broady, K. W., King, G. F., Nicholson, G. M., 2007. The omega-atracotoxins: selective blockers of insect M-LVA and HVA calcium channels. Biochem Pharmacol 74, 623-638.
Cohen, S., Duffus, J. E., Liu, H. Y., 1989. Acquisition, Interference, and Retention of Cucurbit Leaf Curl Viruses in Whiteflies. Phytopathology 79, 109-113.
Czosnek, H., Ghanim, M., Ghanim, M., 2002. The circulative pathway of begomoviruses in the whitefly vector Bemisia tabaci—insights from studies with Tomato yellow leaf curl virus. Ann Appl Biol 140, 215-231.
De Barro, P. J., Liu, S. S., Boykin, L. M., Dinsdale, A. B., 2011. Bemisia tabaci: a statement of species status. Annu Rev Entomol 56, 1-19.
Fauquet, C. M., Briddon, R. W., Brown, J. K., Moriones, E., Stanley, J., Zerbini, M., Zhou, X., 2008. Geminivirus strain demarcation and nomenclature. Archives of Virology 153, 783-821.
Fitches, E. C., Pyati, P., King, G. F., Gatehouse, J. A., 2012. Fusion to Snowdrop Lectin Magnifies the Oral Activity of Insecticidal omega-Hexatoxin-Hv1a Peptide by Enabling Its Delivery to the Central Nervous System. PLOS ONE 7, e39389.
Fletcher, J. I., Smith, R., O'Donoghue, S. I., Nilges, M., Connor, M., Howden, M. E., Christie, M. J., King, G. F., 1997. The structure of a novel insecticidal neurotoxin, omega-atracotoxin-HV1, from the venom of an Australian funnel web spider. Nat Struct Biol 4, 559-566.
Glick, M., Levy, Y., Gafni, Y., 2009. The Viral Etiology of Tomato Yellow Leaf Curl Disease-A Review. Plant. Protect. Sci. 45, 81-97.
Gotz, M., Popovski, S., Kollenberg, M., Gorovits, R., Brown, J. K., Cicero, J. M., Czosnek, H., Winter, S., Ghanim, M., 2012. Implication of Bemisia tabaci heat shock protein 70 in Begomovirus-whitefly interactions. J Virol 86, 13241-13252.
Ha, C., Coombs, S., Revill, P., Harding, R., Vu, M., Dale, J., 2008. Molecular characterization of begomoviruses and DNA satellites from Vietnam: additional evidence that the New World geminiviruses were present in the Old World prior to continental separation. Journal of General Virology 89, 312-326.
Horowitz, A. R., Ishaaya, I., 2004. Biorational insecticides-mechanisms, selectivity and importance in pest management., in: Horowitz, A. R., Ishaaya, I. (Eds.), Insect pest management. Springer, Berlin, pp. 1-28.
Kemmerer, M., Bonning, B. C., 2018. Transcytosis of Junonia coenia densovirus VP4 across the gut epithelium of Spodoptera frugiperda (Lepidoptera: Noctuidae). Insect Sci. doi: 10.1111/1744-7917.12600
King, G. F., Hardy, M. C., 2013. Spider-venom peptides: structure, pharmacology, and potential for control of insect pests. Annu Rev Entomol 58, 475-496.
Kunik, T., Palanichelvam, K., Czosnek, H., Citovsky, V., Gafni, Y., 1998. Nuclear import of the capsid protein of tomato yellow leaf curl virus (TYLCV) in plant and insect cells. Plant J 13, 393-399.
Kunik, T., Salomon, R., Zamir, D., Navot, N., Zeidan, M., Michelson, I., Gafni, Y., Czosnek, H., 1994. Transgenic Tomato Plants Expressing the Tomato Yellow Leaf Curl Virus Capsid Protein Are Resistant to the Virus. Bio-Technol 12, 500-504.
Lapidot, M., Friedmann, M., 2002. Breeding for resistance to whitefly-transmitted geminiviruses. Ann Appl Biol 140, 109-127.
LeOra-Software, 1987. POLO-PC, a user's guide to probit and logit analysis. LeOra Software, Berkeley, California.
Moffat, A. S., 1999. Plant pathology-Geminiviruses emerge as serious crop threat. Science 286, 1835-1835.
Mound, L. A., Halsey, S. H., 1978. Whitefly of the world: a systematic catalogue of the Aleyrodidae (Homoptera) with host plant and natural enemy data. British Museum (Natural History), Chichester, UK.
Muniyappa, V., Jalikop, S. H., Saikia, A. K., Chennarayappa, Shivashankar, G., Bhat, A. I., Ramappa, H. K., 1991. Reaction of Lycopersicon Cultivars and Wild Accessions to Tomato Leaf Curl Virus. Euphytica 56, 37-41.
Nombela, G., Muniz, M., 2010. Host plant resistance for the management of Bemisia tabaci: a multicrop survey with emphasis on tomato, in: Stansly, P. A., Naranjo, S. E. (Eds.), Bemisia: bionomics and management of a global pest. Springer, Dortrecht, pp. 357-383.
Ohnishi, J., Kitamura, T., Terami, F., Honda, K., 2009. A selective barrier in the midgut epithelial cell membrane of the nonvector whitefly Trialeurodes vaporariorum to Tomato yellow leaf curl virus uptake. J Gen Plant Pathol 75, 131-139.
Pal, N., Yamamoto, T., King, G. F., Waine, C., Bonning, B., 2013. Aphicidal efficacy of scorpion- and spider-derived neurotoxins. Toxicon 70C, 114-122.
Palumbo, J. C., Horowitz, A. R., Prabhaker, N., 2001. Insecticidal control and resistance management for Bemisia tabaci. Crop Prot 20, 739-765.
Pan, L. L., Chen, Q. F., Zhao, J. J., Guo, T., Wang, X. W., Hariton-Shalev, A., Czosnek, H., Liu, S. S., 2017. Clathrin-mediated endocytosis is involved in Tomato yellow leaf curl virus transport across the midgut barrier of its whitefly vector. Virology 502, 152-159.
Perring, T. M., Farrar, C. A., Cooper, A. D., Bellows, T. S., Rodriguez, R. J., 1993. Determining Whitefly Species-Response. Science 261, 1334-1335.
Polston, J. E., De Barro, P., Boykin, L. M., 2014. Transmission specificities of plant viruses with the newly identified species of the Bemisia tabaci species complex. Pest Manag Sci 70, 1547-1552.
Prins, M., Laimer, M., Noris, E., Schubert, J., Wassenegger, M., Tepfer, M., 2008. Strategies for antiviral resistance in transgenic plants. Mol Plant Pathol 9, 73-83.
Rosell, R. C., Torres-Jerez, I., Brown, J. K., 1999. Tracing the geminivirus-whitefly transmission pathway by polymerase chain reaction in whitefly extracts, saliva, hemolymph, and honeydew. Phytopathology 89, 239-246.
Rosen, R., Kanakala, S., Kliot, A., Pakkianathan, B. C., Abu Farich, B., Santana-Magal, N., Elimelech, M., Kontsedalov, S., Lebedev, G., Cilia, M., Ghanim, M., 2015. Persistent, circulative transmission of begomoviruses by whitefly vectors. Current Opinion in Virology 15, 1-8.
Shepherd, D. N., Martin, D. P., Thomson, J. A., 2009. Transgenic strategies for developing crops resistant to geminiviruses. Plant Sci 176, 1-11.
Sinisterra, X. H., Polston, J. E., Abouzid, A. M., Hiebert, E., 1999. Tobacco plants transformed with a modified coat protein of tomato mottle begomovirus show resistance to virus infection. Phytopathology 89, 701-706.
Sivakumar, S., Wang, Z., Harrison, R. L., Liu, S., Miller, W. A., Bonning, B. C., 2009. Baculovirus-expressed virus-like particles of Pea enation mosaic virus vary in size and encapsidate baculovirus mRNAs. Virus Res 139, 54-63.
Tedford, H. W., Gilles, N., Menez, A., Doering, C. J., Zamponi, G. W., King, G. F., 2004. Scanning mutagenesis of omega-atracotoxin-Hv1a reveals a spatially restricted epitope that confers selective activity against insect calcium channels. J Biol Chem 279, 44133-44140.
Thompson, W. M. O., 2006. Influence of Amino Acids on Cassava Biotype Bemisia tabaci (Gennadius) (Homoptera: Aleyrodidae) When Feeding on an Artificial System J Entomol 3, 198-203.
Thresh, J. M., Otim-Nape, G. W., Thankappan, M., Muniyappa, V., 1998. The mosaic diseases of cassava in Africa and India caused by whitefly-borne geminiviruses. Rev Plant Pathol 77, 935-945.
Vanderschuren, H., Stupak, M., Futterer, J., Gruissem, W., Zhang, P., 2007. Engineering resistance to geminiviruses—review and perspectives. Plant Biotechnol J 5, 207-220.
Varma, A., Dhar, A. K., Mandal, B., 1992. MYMV transmission and control in India, in: Green, S. K., Doo-Hwan, K. (Eds.), Mungbean yellow mosaic disease. Proceedings of an international workshop, Asian Vegetable Research and Development Center, Taipei, pp. 8-27.
Wang, X., Smith, R., Fletcher, J. I., Wilson, H., Wood, C. J., Howden, M. E., King, G. F., 1999. Structure-function studies of omega-atracotoxin, a potent antagonist of insect voltage-gated calcium channels. Eur J Biochem 264, 488-494.
Wang, Y., Gosselin Grenet, A. S., Castelli, I., Cermenati, G., Ravallec, M., Fiandra, L., Debaisieux, S., Multeau, C., Lautredou, N., Dupressoir, T., Li, Y., Casartelli, M., Ogliastro, M., 2013. Densovirus crosses the insect midgut by transcytosis and disturbs the barrier epithelial function. J Virol. 87 (22), 12380-12391.
Whitfield, A. E., Falk, B. W., Rotenberg, D., 2015. Insect vector-mediated transmission of plant viruses. Virology 479-480, 278-289.
Windley, M. J., Herzig, V., Dziemborowicz, S. A., Hardy, M. C., King, G. F., Nicholson, G. M., 2012. Spider-venom peptides as bioinsecticides. Toxins (Basel) 4, 191-227.
Xia, W. Q., Liang, Y., Chi, Y., Pan, L. L., Zhao, J., Liu, S. S., Wang, X. W., 2018. Intracellular trafficking of begomoviruses in the midgut cells of their insect vector. PLOS Pathog 14, e1006866.
Yang, Y., Sherwood, T. A., Patte, C. P., Hiebert, E., Polston, J. E., 2004. Use of Tomato yellow leaf curl virus (TYLCV) rep gene sequences to engineer TYLCV resistance in tomato. Phytopathology 94, 490-496.
Ffrench-Constant, R. H. et al. (2007) Insecticidal toxins from Photorhabdus bacteria and their potential use in agriculture. Toxicon 49, 436-451Gade, G. (2004). “Regulation of intermediary metabolism and water balance of insects by neuropeptides.” Annual review of entomology 49:93-113.
Schoofs L, Janssen I, Veelaert D, Vanden Broeck J, Tobe S S, De Loof A., Ann N Y Acad Sci. 1998 May 15; 839:301-5.

What is claimed is:

1. A fusion protein comprising a carrier protein attached to a second peptide via a peptide linker, wherein the carrier protein is derived from a Tomato yellow leaf curl virus (TYLCV).

2. The fusion protein of claim 1, wherein the carrier protein is a TYLCV coat protein.

3. The fusion protein of claim 1 or claim 2, wherein the coat protein comprises the amino acid sequence set forth in SEQ ID NO: 2.

4. The fusion protein of claim 3, wherein the coat protein is encoded by a nucleotide sequence set forth in SEQ ID NO: 1.

5. The fusion protein of any one of claims 1-3, wherein the peptide linker is a protease-resistant linker.

6. The fusion protein of claim 5, wherein the peptide linker is encoded by the nucleotide sequence set forth in SEQ ID NO: 3.

7. The fusion protein of any one of claims 1-6, wherein the second peptide comprises an insect-specific toxin.

8. The fusion protein of claim 7, wherein the insect-specific toxin is an insect-specific neurotoxin.

9. The fusion protein of any one of claims 1-8, wherein the insect-specific toxin is an arthropod-derived neuropeptide, enzyme or hormone.

10. The fusion protein of any one of claims 1-9, wherein the insect-specific toxin is Hv1a, proctolin, or Aedes aegypti trypsin modulating oostatic factor (TMOF).

11. The fusion protein of any one of claims 1-9, that is encoded by the nucleotide sequence set forth in SEQ ID NO: 11.

12. The fusion protein of any one of claims 1-10, comprising the amino acid sequence set forth in SEQ ID NO: 12.

13. A composition comprising the fusion protein of any one of claims 1-12 and a suitable carrier or excipient.

14. A nucleotide sequence that encodes the fusion protein of any one of claims 1-12.

15. A vector comprising a nucleotide sequence that encodes the fusion protein of any one of claims 1-12 operably linked to a plant expressible promoter.

16. A vector comprising the nucleotide sequence set forth in SEQ ID NO: 11 operably linked to a plant expressible promoter.

17. The vector of claim 15 or claim 16, wherein the plant expressible promoter is a phloem-specific promoter, a leaf-specific promoter, a light activated promoter or a leaf-damage activated promoter.

18. The vector of claim 15 or claim 16, wherein the plant expressible promoter is a constitutive promoter.

19. A transgenic plant comprising the nucleotide sequence of claim 14.

20. A transgenic plant comprising the nucleotide sequence set forth in SEQ ID NO: 11.

21. A method of producing a transgenic plant comprising introducing the nucleotide sequence of claim 14 or a vector of any one of claims 15-18 into a cell of the plant.

22. A method of producing a transgenic plant comprising introducing the nucleotide sequence of SEQ ID NO: 11 into a cell of the plant.

23. The method of claim 21 or claim 22, comprising introducing the nucleotide sequence into the plant by transformation.

24. The method of any one of claims 21-23, further comprising sexually crossing a plant with the transgenic plant of claim 20 or claim 21.

25. A seed produced by the plant of claim 19 or claim 20.

26. A method of controlling insect pests, the method comprising feeding an insect with a food source comprising a fusion protein as in any one of claims 1-12 or a composition of claim 13, wherein upon ingestion, the fusion protein passes across the gut epithelium and moves into the hemocoel of the insect.

27. The method of claim 26, wherein the insect pest is a whitefly.


SEQUENCE LISTING (24 sequences)

SEQ ID NO: 1 - DNA, 771 bp, Begomovirus plant virus
  source 1..771: mol_type = other DNA
tcgaagcgaccaggcgatataatcatttccacgcccgtctcgaaggttcgccgaaggctgaacttcgacagcccatacagcaaccgtgctgctgtccccattgtccaaggcacaaacaagcgacgatcatggacgtacaggcccatgtaccgaaagcccagaatatacagaatgtatcgaagccctgatgttccccgtggatgtgaaggcccatgtaaagtccagtcttatgagcagcgggatgatattaagcacactggtgttgttcgttgtgttagtgatgttactcgtggatctggaattactcacagagtcggtaagaggttctgtgttaaatcgatatattttttaggtaaagtctggatggatgaaaatatcaagaagcagaatcacactaatcaggtcatgttctttttggtccgtgatagaaggccctatggaagcagtccaatggattttggacaggtttttaatatgttcgataatgagcccagtaccgcaactgtgaagaatgatttgcgtgataggtttcaagtgatgaggaaatttcatgctacagttattggtgggccctctggaatgaaggaacaggcattagttaagagattttttaaaattaacagtcatgtaacttataatcatcaggaggcagccaagtacgagaaccatactgaaaacgccttgttattgtatatggcatgtacgcatgcctctaatccagtgtatgcaactatgaaaatacgcatctatttctatgattcaatatcaaat

SEQ ID NO: 2 - protein, 257 aa, Begomovirus plant virus
SKRPGDIIISTPVSKVRRRLNFDSPYSNRAAVPIVQGTNKRRSWTYRPMYRKPRIYRMYRSPDVPRGCEGPCKVQSYEQRDDIKHTGVVRCVSDVTRGSGITHRVGKRFCVKSIYFLGKVWMDENIKKQNHTNQVMFFLVRDRRPYGSSPMDFGQVFNMFDNEPSTATVKNDLRDRFQVMRKFHATVIGGPSGMKEQALVKRFFKINSHVTYNHQEAAKYENHTENALLLYMACTHASNPVYATMKIRIYFYDSISN

SEQ ID NO: 3 - DNA, 84 bp, synthetic construct
  misc_feature 1..84: note = Synthetic
  source 1..84: mol_type = other DNA
ggggacgacgctcccccgtcaccagggcctgatcccgggccccaaccaccaccacctccacccccaagtcccactcccgtagga

SEQ ID NO: 4 - protein, 28 aa, synthetic construct
  REGION 1..28: note = Synthetic
GDDAPPSPGPDPGPQPPPPPPPSPTPVG

SEQ ID NO: 5 - DNA, 705 bp, synthetic construct
  misc_feature 1..705: note = Synthetic
  source 1..705: mol_type = other DNA
gtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 6 - DNA, 1560 bp, synthetic construct
  misc_feature 1..1560: note = synthetic
  source 1..1560: mol_type = other DNA
tcgaagcgaccaggcgatataatcatttccacgcccgtctcgaaggttcgccgaaggctgaacttcgacagcccatacagcaaccgtgctgctgtccccattgtccaaggcacaaacaagcgacgatcatggacgtacaggcccatgtaccgaaagcccagaatatacagaatgtatcgaagccctgatgttccccgtggatgtgaaggcccatgtaaagtccagtcttatgagcagcgggatgatattaagcacactggtgttgttcgttgtgttagtgatgttactcgtggatctggaattactcacagagtcggtaagaggttctgtgttaaatcgatatattttttaggtaaagtctggatggatgaaaatatcaagaagcagaatcacactaatcaggtcatgttctttttggtccgtgatagaaggccctatggaagcagtccaatggattttggacaggtttttaatatgttcgataatgagcccagtaccgcaactgtgaagaatgatttgcgtgataggtttcaagtgatgaggaaatttcatgctacagttattggtgggccctctggaatgaaggaacaggcattagttaagagattttttaaaattaacagtcatgtaacttataatcatcaggaggcagccaagtacgagaaccatactgaaaacgccttgttattgtatatggcatgtacgcatgcctctaatccagtgtatgcaactatgaaaatacgcatctatttctatgattcaatatcaaatggggacgacgctcccccgtcaccagggcctgatcccgggccccaaccaccaccacctccacccccaagtcccactcccgtaggagtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 7 - DNA, 111 bp, Hadronyche versuta
  source 1..111: mol_type = other DNA
agcccgacctgcatcccgagcggtcagccgtgcccgtacaacgaaaactgctgcagccagagctgcaccttcaaagaaaacgaaaacggtaacaccgtgaaacgttgcgat

SEQ ID NO: 8 - protein, 37 aa, Hadronyche versuta
SPTCIPSGQPCPYNENCCSQSCTFKENENGNTVKRCD

SEQ ID NO: 9 - DNA, 111 bp, synthetic construct
  misc_feature 1..111: note = Synthetic
  source 1..111: mol_type = other DNA
agcccgacctgcatcccgagcggtcagccgtgcccgtacaacgaaaactgctgcagccagagctgcaccttcaaagaagcggaaaacggtaacaccgtgaaagcgtgcgat

SEQ ID NO: 10 - protein, 37 aa, synthetic construct
  REGION 1..37: note = Synthetic
SPTCIPSGQPCPYNENCCSQSCTFKEAENGNTVKACD

SEQ ID NO: 11 - DNA, 966 bp, synthetic construct
  misc_feature 1..966: note = Synthetic
  source 1..966: mol_type = other DNA
tcgaagcgaccaggcgatataatcatttccacgcccgtctcgaaggttcgccgaaggctgaacttcgacagcccatacagcaaccgtgctgctgtccccattgtccaaggcacaaacaagcgacgatcatggacgtacaggcccatgtaccgaaagcccagaatatacagaatgtatcgaagccctgatgttccccgtggatgtgaaggcccatgtaaagtccagtcttatgagcagcgggatgatattaagcacactggtgttgttcgttgtgttagtgatgttactcgtggatctggaattactcacagagtcggtaagaggttctgtgttaaatcgatatattttttaggtaaagtctggatggatgaaaatatcaagaagcagaatcacactaatcaggtcatgttctttttggtccgtgatagaaggccctatggaagcagtccaatggattttggacaggtttttaatatgttcgataatgagcccagtaccgcaactgtgaagaatgatttgcgtgataggtttcaagtgatgaggaaatttcatgctacagttattggtgggccctctggaatgaaggaacaggcattagttaagagattttttaaaattaacagtcatgtaacttataatcatcaggaggcagccaagtacgagaaccatactgaaaacgccttgttattgtatatggcatgtacgcatgcctctaatccagtgtatgcaactatgaaaatacgcatctatttctatgattcaatatcaaatggggacgacgctcccccgtcaccagggcctgatcccgggccccaaccaccaccacctccacccccaagtcccactcccgtaggaagcccgacctgcatcccgagcggtcagccgtgcccgtacaacgaaaactgctgcagccagagctgcaccttcaaagaaaacgaaaacggtaacaccgtgaaacgttgcgat

SEQ ID NO: 12 - protein, 322 aa, synthetic construct
  REGION 1..322: note = Synthetic
SKRPGDIIISTPVSKVRRRLNFDSPYSNRAAVPIVQGTNKRRSWTYRPMYRKPRIYRMYRSPDVPRGCEGPCKVQSYEQRDDIKHTGVVRCVSDVTRGSGITHRVGKRFCVKSIYFLGKVWMDENIKKQNHTNQVMFFLVRDRRPYGSSPMDFGQVFNMFDNEPSTATVKNDLRDRFQVMRKFHATVIGGPSGMKEQALVKRFFKINSHVTYNHQEAAKYENHTENALLLYMACTHASNPVYATMKIRIYFYDSISNGDDAPPSPGPDPGPQPPPPPPPSPTPVGSPTCIPSGQPCPYNENCCSQSCTFKENENGNTVKRCD

SEQ ID NO: 13 - DNA, 26 bp, synthetic construct
  misc_feature 1..26: note = Synthetic
  source 1..26: mol_type = other DNA
ataggatcctatatgtcgaagcgacc

SEQ ID NO: 14 - DNA, 44 bp, synthetic construct
  misc_feature 1..44: note = Synthetic
  source 1..44: mol_type = other DNA
tggtgacgggggagcgtcgtccccatttgatattgaatcataga

SEQ ID NO: 15 - DNA, 44 bp, synthetic construct
  misc_feature 1..44: note = Synthetic
  source 1..44: mol_type = other DNA
ctatttctatgattcaatatcaaatggggacgacgctcccccgt

SEQ ID NO: 16 - DNA, 27 bp, synthetic construct
  misc_feature 1..27: note = Synthetic
  source 1..27: mol_type = other DNA
cgcgaattcgcgttagccgctttactt

SEQ ID NO: 17 - DNA, 37 bp, synthetic construct
  misc_feature 1..37: note = Synthetic
  source 1..37: mol_type = other DNA
ggttccgcgtggatccactggggacgacgctcccccg

SEQ ID NO: 18 - DNA, 49 bp, synthetic construct
  misc_feature 1..49: note = Synthetic
  source 1..49: mol_type = other DNA
gatgcggccgctcgagactttagccgctttacttgtacagctcgtccat

SEQ ID NO: 19 - DNA, 37 bp, synthetic construct
  misc_feature 1..37: note = Synthetic
  source 1..37: mol_type = other DNA
ttagaattcctttcgaagcgaccaggcgatataatca

SEQ ID NO: 20 - DNA, 44 bp, synthetic construct
  misc_feature 1..44: note = Synthetic
  source 1..44: mol_type = other DNA
gttatcctcctcgcccttgctcactcctacgggagtgggacttg

SEQ ID NO: 21 - DNA, 44 bp, synthetic construct
  misc_feature 1..44: note = Synthetic
  source 1..44: mol_type = other DNA
caagtcccactcccgtaggagtgagcaagggcgaggaggataac

SEQ ID NO: 22 - DNA, 44 bp, synthetic construct
  misc_feature 1..44: note = Synthetic
  source 1..44: mol_type = other DNA
ataagcataaagcttattttacttgtacagctcgtccatgccgc

SEQ ID NO: 23 - DNA, 20 bp, synthetic construct
  misc_feature 1..20: note = Synthetic
  source 1..20: mol_type = other DNA
ttagaattcctttcgaagcg

SEQ ID NO: 24 - DNA, 27 bp, synthetic construct
  misc_feature 1..27: note = Synthetic
  source 1..27: mol_type = other DNA
ataagcataaagcttattttacttgta